(19) United States
(12) Patent Application Publication
(10) Pub. No.: US 2007/0218497 A1
(43) Pub. Date: Sep. 20, 2007

(54) Quick Test for the Diagnosis of Alzheimer's Disease

(76) Inventors: Thomas Arendt, Leipzig (DE); Jens Stieler, Dresden (DE)

Correspondence Address: INTELLECTUAL PROPERTY / TECHNOLOGY LAW, PO BOX 14329, RESEARCH TRIANGLE PARK, NC, 27709, US

(21) Appl. No.: 10/576,142

(22) PCT Filed: Sep. 29, 2004
(86) PCT No.: PCT/EP04/10889

§ 371 Date: Dec. 6, 2006

(30) Foreign Application Priority Data
Oct. 22, 2003 (DE) 10349162.7

(51) Int. Cl. G01N 33/68

(52) U.S. Cl. 435/7.1

(57) ABSTRACT

The invention relates to method for the diagnosis of Alzheimer's disease or the early stages thereof or a predisposition to said disease. Said method is based on quantitative determination of a mitogenically expressible surface marker, in particular CD69, and peripherally accessible cells, e.g. skin cells or lymphocytes, (a) prior to and (b) after mitogenic stimulation. A specific stimulation index a:b is an indication of Alzheimer's disease or early stages thereof or of a predisposition to said disease. The invention also relates to kits which are suitable for carrying out the inventive method of diagnosis.

The present invention relates to a method of diagnosing Alzheimer's disease or an early stage of or predisposition for this disease, which method is based on the quantification of mitogenically expressible surface markers, preferably CD69, of peripherally accessible cells, e.g. skin cells or lymphocytes, (a) before and (b) a) after mitogenic stimulation, a special stimulation index a:b being a sign of Alzheimer's disease or an early stage of or a predisposition for this disease. The present invention also relates to kits suited to carry out the diagnostic method according to the invention.

Alzheimer's disease cannot be diagnosed with ultimate certainty by clinical means and the available paraclinical methods and methods based on apparatus and technology as such. It always requires autopsy verification. The diagnostic differentiation with respect to other demential causes is often difficult, in particular in the early stages of the disease. In these very early stages of the disease, however, assured diagnosis is important for two reasons. On the one hand, it permits the diagnostic differentiation of potentially treatable forms of dementia and thus can subject them to an effective treatment and, on the other hand, it is a precondition for any form of therapeutic intervention in the neurodegenerative process of Alzheimer's disease, which can only be successful in these early stages. Such a diagnostic certainty can only be guaranteed by biomarkers of Alzheimer's disease, i.e. by easily determinable biological changes with sensitivity and specificity adequate for this disease.

Biomarkers of Alzheimer's disease are thus of diagnostic value and shall in particular assist in safely identifying risk groups and patients in preclinical stages and early clinical stages. Biomarkers also serve the follow-up and thus the prognosis and control of the responsiveness to therapeutic interventions. Model biomarkers should comply with certain theoretical and practical requirements. They include in particular a high specificity and sensitivity, the ability to identify preclinical stages, and a high positive and negative predictive value. The biomarkers should be determined, if possible, in a non-invasive way and neither burden nor frighten the patient. The analyses should be inexpensive and adapted to be carried out readily and, if possible, in a family physician's practice. Unfortunately, none of the presently known biomarkers of Alzheimer's disease complies with the above mentioned requirements. In particular on account of the minor sensitivity and specificity of the known biomarkers they are unsuited as diagnostic means. Other diagnostic examinations having greater sensitivity and specificity call for complicated technical preconditions and are thus not suited for a local use with a major group of patients.

Thus, the invention is substantially based on the technical problem of providing a simple method for the diagnosis of Alzheimer's disease, which permits the diagnosis of Alzheimer's disease, the detection of preclinical disease stages and the diagnostic differentiation of Alzheimer's disease from other dementias with adequate sensitivity and specificity.

This technical problem was solved by providing the embodiments characterized in the claims.

It was possible to develop a diagnostic method which is based on the determination of the mitogenic index (activation index) using peripherally accessible patient cells, such as skin cells or blood lymphocytes, with and without mitogenic stimulation, e.g. after immunomagnetic cell separation. The activation of these cells is accompanied by the surface presentation of activation markers which can be quantitatively detected, preferably by means of antigen-antibody interactions, magnetic particles preferably coated with antibodies being used, which permits the magnetic cell separation and subsequently the quantification of the number of cells bearing this surface marker before and after mitogenic stimulation. This feature shows disease-specific deviations from the normal findings. The method according to the invention thus permits the diagnosis of Alzheimer's disease, the detection of preclinical disease stages and the diagnostic differentiation of Alzheimer's disease from other dementias.

Thus, the present invention relates to a method of diagnosing Alzheimer's disease or an early stage of or a predisposition for this disease by means of a patient sample, this method comprising the steps of:
(a) mitogenic stimulation of the peripherally accessible cells in the sample;
(b) quantification of the mitogenically stimulated cells within the cell population before and after step (a) by means of one or more surface markers expressed after mitogenic stimulation, the cells bearing surface markers being separated from the cells bearing no surface markers using antibodies directed against the surface markers; and
(c) determination of the stimulation index as a relationship between the number of cells bearing the surface marker or markers before and after step (a),
   a stimulation index which reaches at least 10 times, as a maximum 100 times, the unstimulated control sample, being a sign of Alzheimer's disease or an early stage of or a predisposition for this disease.

A person skilled in the art knows suitable measures serving for obtaining patient samples suited for the method according to the invention, which contain sufficient mitogenically stimulable cells. For example, suitable samples are dermal tissue samples, blood samples, preferably from venous blood, cells from the liquor cerebrospinalis, and cells from the urine.

In a preferred embodiment of the diagnostic method according to the invention, e.g. when a blood sample is used, an anticoagulative compound, e.g. sodium citrate or heparin, is added for the purpose of stabilization prior to the other method steps.

The term “diagnosis of Alzheimer's disease” as used herein also comprises the follow-up and thus the prognosis, the control of the efficiency of therapeutic interventions and the diagnostic differentiation of the disease from other dementias.

The term “peripherally accessible cells” as used herein refers to cells which can be removed without an operation or in a (minimally) invasive fashion from the human organism and they comprise e.g. skin cells and lymphocytes of the peripheral blood, the latter being preferred for the method according to the invention.

The mitogenic stimulation for obtaining the expression of surface markers can be effected by known stimulators, such as phytohemagglutinin (PHA), protein A, PWM or other compounds having a trophic or mitogenic effect. The stimulation can be effected by adding the individual compounds or by a combined addition.

The person skilled in the art knows suitable experimental conditions for such a stimulation, e.g. as regards the concentration of the mitogens used, the duration of stimulation and other incubation conditions. The stimulation should be carried out in suitable vessels permitting adequate gas exchange. The concentrations of the respective stimulation agents should be within the physiological range which is 1 μg/ml to 20 μg/ml for PHA, 1 μg/ml to 50 μg/ml for PWM, and 10 μg/ml to 200 μg/ml for protein A. The stimulation period depends on the expression rate of the molecule to be examined. However, stimulation periods of 2 to 24 hours may be necessary for certain examinations. In the case of CD69 a stimulation period of 4 hours is optimum. Stimulation should be carried out under physiological conditions and it can be conducted in a gassing incubator at 37° C. and with 5% CO2, for example.

The person skilled in the art also knows suitable surface markers by means of which a mitogenic stimulation manifests itself, e.g. CD69, CD25, CD45RO, CD63 and HLA-Dr, the surface marker CD69 being preferred. For the purposes of the invention, it is also possible to carry out a determination of a combination of surface markers or the further specification of the cells separated by means of a certain surface marker, e.g. CD69, as regards further subpopulations, e.g. by means of (e.g. CD4⁺ and/or CD8⁺ and/or CD19⁺ and/or CD56⁺) subpopulations.

The stimulation index (activation index) follows from the relationship of the number of cells bearing the surface marker or markers before and after the stimulation. A stimulation index which reaches at least 10 times, as a maximum 100 times, the unstimulated control sample, is a sign of an Alzheimer's disease or an early stage of or a predisposition for this disease. A stimulation index which is less than 10 times the unstimulated control sample is no sign of an Alzheimer's disease or an early stage of or a predisposition for this disease. The cells bearing the surface markers can be determined according to conventional methods, e.g. Western blot, ELISA, RIA, FACS, LSC, etc.

In order to determine the cells bearing the surface markers, they are preferably separated from the cells bearing no surface marker or bearing other surface markers by means of characteristic cell features.

In the diagnostic method of the present invention, the cells bearing the surface markers are separated from the cells which bear no surface markers by antibodies directed against the desired surface marker(s). The antibodies suited for this purpose may be monoclonal, polyclonal or synthetic antibodies or fragments thereof. In this connection, the term “fragment” means all the parts of the monoclonal antibody (e.g. Fab, Fv or single chain Fv fragments) which have an epitope specificity the same as that of the complete antibody. The production of such fragments is known to the person skilled in the art, many antibodies directed against surface markers are also commercially available.

In the most preferred embodiment of the diagnostic method according to the invention, the antibody or antibodies specific to surface markers are bound to magnetic particles, e.g. paramagnetic beads (e.g. available from DYNAL A.S., P.O. Box 158 Skøyen, N-0212 Oslo, Norway), which permits the separation of the cells with the corresponding surface markers via immunomagnetic separation according to current methods.

The stimulation index can then be specified by determining the amount of cells separated by means of the desired surface marker on the basis of its nucleic acid content and/or protein content using current methods, e.g. after lysis of the cells by spectrophotometric determination of the nucleic acid or protein content or after staining the nucleic acid using specific dyes, e.g. ethidium bromide, propidium iodide, acridine orange, DAPI, etc., by means of photometric quantification. The cell number can be calculated from the protein and/or nucleic acid content of the sample by means of calibration curves.

The present invention also relates to a kit which is suited for carrying out the diagnostic method according to the invention and contains at least the following components:
(a) a compound for mitogenic stimulation;
(b) at least one antibody directed against a surface marker expressed after mitogenic stimulation, preferably an antibody bound to a magnetic particle.

The kit according to the invention also preferably contains
(a) at least one reaction vessel;
(b) an anticoagulative compound and/or a buffer for cell lysis;
(c) a buffer for fixing the cells;
(d) substances required for the quantification of the DNA and/or protein concentration and ready-made solutions for the production of a calibration curve;
(e) a magnet for separating the cells bound to the magnetic particles (contained if an antibody bound to a magnetic particle is used); and
(f) a reagent for removing bound magnetic particles (contained if an antibody bound to a magnetic particle is used).

In a preferred embodiment of the kit according to the invention, the antibody is an anti-CD69 antibody. Moreover, the kit can additionally contain, or contain instead of the anti-CD69 antibody, an anti-CD4 and/or anti-CD8 antibody.

Finally, the kit according to the invention may be present, where appropriate, in combination with one or more suitable further detection agents, e.g. fluorescence-coupled primary antibodies, secondary antibodies, detection agents for proteins and/or nucleic acids, e.g. an intercalating dye, etc.

### EXAMPLE

### Determination of the Mitogenic Stimulation Index by Means of CD69 in Patients Suffering from Alzhelmer's Disease

The determination of features known to date of Alzheimer's disease, which can be carried out in living patients (biomarkers), only shows insufficient sensitivity and specificity or is not suited for examinations with great case numbers for reasons of cost or the highly complicated test arrangement. With clinical means, the diagnostic certainty is only 80% to 90% and difficult in particular in the early stages of the disease as regards diagnostic differentiation. The detection of preclinical disease stages is currently not possible for lack of a suitable biomarker.

The neurodegenerative changes are based on disturbed processes of the intracellular mediation of trophic and mitogenic signals in the case of Alzheimer's disease. These dysfunctions of intracellular signal transduction are not limited to the nervous system. They can similarly also be found on skin cells and lymphocytes of the peripheral blood of these patients. On account of their disease specificity, this alteration is of diagnostic value and suited as a biomarker.

In the below example, the question of whether there is a dysfunction typical of Alzheimer's disease of the intracellular mediation of trophic and mitogenic signals was determined by immunomagnetic cell separation of CD69 presenting lymphocytes before and after mitogenic stimulation.

The blood is collected by venous puncture using a blood withdrawal system from SARSTEDT company. The blood is here stabilized during the withdrawal by anticoagulants integrated into the blood withdrawal system, such as sodium citrate or sodium heparin. In this form, it can be stored at room temperature for 24 to 48 hours. The stimulation experiments were carried out in reaction vessels which can well be aerated, such as a 24 well suspension culture plate of the company Greiner bio-one. For this, the mitogens phytohemagglutinin (PHA), protein A and pokeweed mitogen (PWM) were used separately or in different combinations for 400 μl stabilized whole blood each. The final concentrations of the respective mitogens were within the physiological range and were 12 μg/mI for PHA, 50 μg/ml for protein A and 4 μg/ml for PWM in this example. The stimulation was carried out under physiological conditions at 37° C. and a CO₂ concentration of 5% in a gassing incubator for 4 hours. 100 μl each of the stimulated whole blood was incubated with different antibody coated magnetic particles. In this example, anti-CD4 and anti-CD8 coated magnetic particles from DYNAL company were used. The corresponding magnetic particles were added to the particular sample in excess (10 μl magnetic particle suspension) to ensure complete isolation of the corresponding lymphocyte subpopulation. Following an incubation period of 30 minutes at 4° C., the corresponding lymphocyte subpopulation was separated magnetically and after subsequent wash steps converted into 100 μl defined medium, in this example RPMI1640, mixed with 1% fetal calf serum (FCS). The bound magnetic particles were removed in this example using 10 μl DETACHaBEAD of DYNAL company each. Following an incubation period of 45 minutes at room temperature, the removed magnetic particles were separated and the cell suspension was taken up in a defined medium, in this example RPMI1640, after several wash steps. By the addition of a specific lysis buffer, the cells were broken up, the DNA was labelled with specific DNA dyes, such as ethidium bromide, propidium iodide, acridine orange or DAPI, and subsequently quantified photometrically. The protein content of the samples was compared by means of the protein determination method according to Bradford. The cell number was calculated from the DNA and/or protein content of the sample by means of calibration curves. This procedure permitted a direct conclusion about the cell number. The calculation of the quotient from the number of CD69 presenting cells before and after mitogenic stimulation (stimulation index) furnished information on alterations of the mitogenic stimulability of these cells.

A stimulation index which reaches at least 10 times, as a maximum 100 times, the unstimulated control sample, is a sign of an Alzheimer's disease or an early stage of or a predisposition for this disease. A stimulation index which is less than 10 times the unstimulated control sample is no sign of an Alzheimer's disease or an early stage of or a predisposition for this disease.

In another experiment, the protein content of the sample was determined and the DNA content was determined without the addition of DNA-staining substances for the quantification of the CD69 presenting cells. In this case, the absorption of light having a certain wavelength (e.g. 260 nm or 280 nm) by DNA or protein was measured.

1. A method of diagnosing Alzheimer's disease or an early stage of or a predisposition for this disease by means of a patient sample, the method comprising the steps of:
(a) mitogenic stimulation of the peripherally accessible cells in the sample;
(b) quantification of the mitogenically stimulated cells within the cell population before and after step (a) by means of one or more surface markers expressed after mitogenic stimulation, the cells bearing the surface markers being separated from the cells bearing no surface markers by means of antibodies directed against the surface markers;
(c) determination of the stimulation index as a relationship of the number of cells bearing the surface marker or markers before or after step (a).

2. The method according to claim 1, wherein the sample is a blood sample and the cells are lymphocytes.

3. The method according to claim 1, wherein the surface marker is CD69.

4. The method according to claim 3, wherein the CD69⁺ cells are further specified with respect to CD4⁺ and/or CD8⁺ subpopulations.

5. The method according to claim 1, wherein the blood is stabilized by one or more anticoagulative compounds before step (a).

6. The method according to claim 1, wherein the cells are stimulated by PHA, protein A or PWM.

7. The method according to claim 1, wherein the antibodies in step (b) are bound to magnetic particles and the separation is carried out via immunomagnetic separation.

8. The method according to claim 1, wherein the stimulation index is determined by determining the protein content and/or nucleic acid content of the cells bearing surface markers before and after step (a).

9. A kit for the diagnosis of Alzheimer's disease or an early stage of or a predisposition for this disease, the kit containing the following constituents:
(a) a compound for mitogenic stimulation; and
(b) at least one antibody directed against a surface marker expressed after mitogenic stimulation.

10. The kit according to claim 9, also containing:
(a) an anticoagulative compound; and/or
(b) a buffer for cell lysis.

11. The kit according to claim 9, wherein the antibody is an antibody bound to a magnetic particle.

12. The kit according to claim 9, wherein the antibody is an anti-CD69 antibody.

13. The kit according to claim 9, which also contains an anti-CD4 and/or CD8 antibody.
